(19) United States
(12) Patent Application Publication
(10) Pub. No.: US 2008/0262029 A1
(43) Pub. Date: Oct. 23, 2008

(54) Acid and Base Salt Forms of Gaboxadol

(75) Inventors: Louis S. Crocker, Belle Mead, NJ (US); Jerry A. Murry, Newbury Park, CA (US); Karthik Nagapudi, Woodbridge, NJ (US); Kara Beth Rubin, Jackson, NJ (US)

Correspondence Address: DARBY & DARBY P.C., P.O. BOX 770, Church Street Station, New York, NY, 10008-0770, US

(73) Assignee: H. Lundbeck A/S, Valby-Copenhagen (DK)

(21) Appl. No.: 11/912,339

(22) PCT Filed: Apr. 25, 2006
(86) PCT No.: PCT/US2006/015789

§ 371 Date: Jul. 3, 2008

Related U.S. Application Data
(60) Provisional application No. 60/676,332, filed on Apr. 29, 2005.

(51) Int. Cl. A61K 31/437; C07D 498/04; A61P 25/00

(52) U.S. Cl. 514/302; 546/116

(57) ABSTRACT

The present invention is directed to novel acid salt forms and base salt forms of the compound gabaoxadol (4,5,6,7-tetrahydroisoxazolo[5,4-c]pyridin-3-ol) and hydrates, solvates and polymorphic forms thereof. The invention is further concerned with pharmaceutical compositions containing the salt forms as an active ingredient, methods for treatment of disorders susceptible to amelioration by GABAA receptor agonism with the salt forms, and processes for the preparation of the salt forms.

### BACKGROUND OF THE INVENTION

The compound 4,5,6,7-tetrahydroisoxazolo[5,4-c]pyridin-3-ol (also known as THIP or gaboxadol, and hereinafter referred to as gaboxadol) is a GABA_{A} receptor agonist (see, for example, EP 0 000 338) and has therefore been suggested for use in treating a variety of neurological and psychiatric disorders such as epilepsy, Parkinson's disease, schizophrenia and Huntingdon's chorea. More recently, there has been disclosed the use of gaboxadol for treatment of sleep disorders (WO 97/02813) and premenstrual syndrome (WO 02/40009), and the disclosure that gaboxadol is a particularly potent agonist at GABA_{A} receptors comprising α4 and δ subunits (Brown et al, British J. Pharmacol., 136, 965-74 (2002). Other indications for which gaboxadol may be suitable include hearing disorders, vestibular disorders, attention deficit hyperactivity disorder, intention tremor and restless leg syndrome.

The preparation of gaboxadol is disclosed in EP 0 000 338, both as the free base and as an acid addition salt, specifically, the hydrobromide salt. Gaboxadol is sold commercially (eg. by Sigma) in the form of the hydrochloride salt, and WO 01/22941 and WO 02/094225 disclose granulated pharmaceutical compositions comprising gaboxadol in the form of the hydrochloride salt.

As detailed in WO 02/094255, use of acid addition salts of gaboxadol such as the hydrochloride in the manufacture of pharmaceutical oral dosage forms such as tablets gives rise to corrosion problems when conventional techniques and equipment are employed. There is therefore a need for novel salt forms of gaboxadol having greater stability and suitability for incorporation in pharmaceutical oral dosage formulations.

### SUMMARY OF THE INVENTION

The present invention is directed to novel acid salt forms and base salt forms of the compound 4,5,6,7-tetrahydroisoxazolo[5,4-c]pyridin-3-ol, and hydrates, solvates and polymorphic forms thereof. The invention is further concerned with pharmaceutical compositions containing the salt forms as an active ingredient, methods for treatment of disorders susceptible to amelioration by GABAA receptor agonism with the salt forms, and processes for the preparation of the salt forms.

### DETAILED DESCRIPTION OF THE INVENTION

The present invention is directed to acid salt forms and base salt forms of the compound gaboxadol, and hydrates, solvates and polymorphic forms thereof.

In one embodiment, the present invention is directed to acid salt forms of gaboxadol, and hydrates, solvates and polymorphic forms thereof.

Within this embodiment, the present invention is directed to an acid salt form of gaboxadol wherein the acid is an inorganic acid or an organic acid, other than hydrochloric acid or hydrobromic acid.

Within this embodiment, the present invention is directed to an acid salt form of gaboxadol wherein the acid is an inorganic acid or an organic acid selected from: acetic, benzenesulfonic, benzoic, camphorsulfonic, citric, ethanesulfonic, fumaric, gluconic, glutamic, isethionic, lactic, maleic, malic, mandelic, methanesulfonic, mucic, nitric, pamoic, pantothenic, phosphoric, succinic, sulfuric, tartaric, and p-toluenesulfonic acid.

Within this embodiment, the present invention is directed to an acid salt form of gaboxadol wherein the acid is selected from: acetic acid, citric acid, fumaric acid, phosphoric acid, tartaric acid, succinic acid and sulfuric acid.

Within this embodiment, the present invention is directed to an acid salt form of gaboxadol which is selected from: gaboxadol acetate, gaboxadol citrate, gaboxadol fumarate, gaboxadol phosphate, gaboxadol tartrate, gaboxadol succinate, gaboxadol sulfate and gaboxadol bis-sulfate, or a hydrate, solvate or polymorphic form thereof.

Further within this embodiment, the present invention is directed to an acid salt form of gaboxadol in crystalline form. Within this embodiment, the present invention is directed to gaboxadol acetate in crystalline form, and hydrates, solvates and polymorphic forms thereof. Within this embodiment, the present invention is directed to gaboxadol citrate in crystalline form, and hydrates, solvates and polymorphic forms thereof. Within this embodiment, the present invention is directed to gaboxadol fumarate in crystalline form, and hydrates, solvates and polymorphic forms thereof. Within this embodiment, the present invention is directed to gaboxadol phosphate in crystalline form, and hydrates, solvates and polymorphic forms thereof. Within this embodiment, the present invention is directed to gaboxadol tartrate in crystalline form, and hydrates, solvates and polymorphic forms thereof. Within this embodiment, the present invention is directed to gaboxadol sulfate in crystalline form, and hydrates, solvates and polymorphic forms thereof. Within this embodiment, the present invention is directed to gaboxadol bis-sulfate in crystalline form, and hydrates, solvates and polymorphic forms thereof.

The present invention is directed to base salt forms of the compound gaboxadol, and hydrates, solvates and polymorphic forms thereof.

Within this embodiment, the present invention is directed to an base salt form of gaboxadol wherein the base is an inorganic base or an organic base.

Within this embodiment, the present invention is directed to an base salt form of gaboxadol wherein the base is an inorganic base or an organic base selected from: aluminum, ammonium, calcium, copper, ferric, ferrous, lithium, magnesium, manganic salts, manganous, potassium, sodium, zinc bases, and primary, secondary, and tertiary amines, substituted amines including naturally occurring substituted amines, cyclic amines, and basic ion exchange resins, such as arginine, betaine, caffeine, choline, N,N-dibenzyl(ethylene)-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine.

Within this embodiment, the present invention is directed to a base salt form of gaboxadol wherein the base is selected from: calcium hydroxide, potassium hydroxide, magnesium hydroxide, sodium hydroxide, choline hydroxide, L-lysine and N,N-dibenzyl(ethylene)diamine.

Within this embodiment, the present invention is directed to a base salt form of gaboxadol which is selected from: gaboxadol calcium, gaboxadol potassium, gaboxadol magnesium, gaboxadol sodium, gaboxadol choline, gaboxadol L-lysine and gaboxadol N,N-dibenzyl(ethylene)diamine, or a hydrate, solvate or polymorphic form thereof.

Further within this embodiment, the present invention is directed to a base salt form of gaboxadol in crystalline form. Within this embodiment, the present invention is directed to gaboxadol calcium in crystalline form, and hydrates, solvates and polymorphic forms thereof. Within this embodiment, the present invention is directed to gaboxadol potassium in crystalline form, and hydrates, solvates and polymorphic forms thereof. Within this embodiment, the present invention is directed to gaboxadol magnesium in crystalline form, and hydrates, solvates and polymorphic forms thereof. Within this embodiment, the present invention is directed to gaboxadol sodium in crystalline form, and hydrates, solvates and polymorphic forms thereof. Within this embodiment, the present invention is directed to gaboxadol choline in crystalline form, and hydrates, solvates and polymorphic forms thereof. Within this embodiment, the present invention is directed to gaboxadol L-lysine in crystalline form, and hydrates, solvates and polymorphic forms thereof. Within this embodiment, the present invention is directed to gaboxadol N,N-dibenzyl(ethylene)diamine in crystalline form, and hydrates, solvates and polymorphic forms thereof.

For the avoidance of any doubt, “gaboxadol” as used herein refers to 4,5,6,7-tetrahydroisoxazolo[5,4-c]pyridin-3-ol free base, which is believed to exist as the zwitterion:

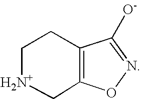

These salt forms of gaboxadol are suitable for incorporation in pharmaceutical formulations and may be incorporated in conventional oral dosage formulations such as tablets using conventional techniques and equipment without the risk of corrosion. The novel salt forms exhibit thermodynamic stability greater than other known salt forms. Utilization of such salt forms would improve the stability of formulated pharmaceutical product. Furthermore, in view of their significant degree of solubility in water, the novel salts are expected to show bioavailability equivalent to that of the acid addition salts previously used for this purpose. These salt forms have superior properties over other forms of the compound in that it they are more suitable for inclusion in pharmaceutical formulations

According to a further aspect of the invention there is provided a pharmaceutical composition comprising, in a pharmaceutically acceptable carrier, a compound selected from: gaboxadol acetate, gaboxadol citrate, gaboxadol fumarate, gaboxadol phosphate, gaboxadol tartrate, gaboxadol succinate, gaboxadol sulfate and gaboxadol bis-sulfate, gaboxadol calcium, gaboxadol potassium, gaboxadol magnesium, gaboxadol sodium, gaboxadol choline, gaboxadol L-lysine and gaboxadol N,N-dibenzyl(ethylene)diamine, or a hydrate, solvate or polymorphic form thereof.

The pharmaceutical composition of this invention is a pharmaceutical preparation, for example, in solid, semisolid or liquid form, which contains one or more of the compounds of the present invention as an active ingredient in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea and other carriers suitable for use in manufacturing preparations in solid, semisolid, or liquid form, and in addition auxiliary, stabilizing, thickening and coloring agents and perfumes may be used. The active object compound is included in the pharmaceutical composition in an amount sufficient to produce the desired effect upon the process or condition of the disease.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Compositions for inhalation or insufflation include suspensions in pharmaceutically acceptable aqueous or organic solvents, or mixtures thereof, and powders. The liquid or solid compositions may contain suitable pharmaceutically acceptable excipients as set out above. Such compositions are administered by the oral or nasal respiratory route for local or systemic effect. Suspension or powder compositions may be administered, preferably orally or nasally, from devices which deliver the formulation in an appropriate manner. The pharmaceutical composition of the invention is preferably in a form suitable for oral administration, such as tablets or capsules. Methods and materials for the formulation of active ingredients as pharmaceutical compositions are well known to those skilled in the art, e.g. from texts such as Remington's Pharmaceutical Sciences (Mack Publishing, 1990).

Gaboxadol acid salts and gaboxadol base salts in accordance with the invention are useful in therapeutic treatment of the human body, and in particular the treatment of disorders susceptible to amelioration by GABAA receptor agonism.

Accordingly, the invention further provides a method of treating disorders susceptible to amelioration by GABAA receptor agonism comprising administering to a patient in need thereof a therapeutically effective amount of a compound selected from: gaboxadol acetate, gaboxadol citrate, gaboxadol fumarate, gaboxadol phosphate, gaboxadol tartrate, gaboxadol succinate, gaboxadol sulfate and gaboxadol bis-sulfate, gaboxadol calcium, gaboxadol potassium, gaboxadol magnesium, gaboxadol sodium, gaboxadol choline, gaboxadol L-lysine and gaboxadol N,N-dibenzyl(ethylene)diamine, or a hydrate, solvate or polymorphic form thereof.

The invention further provides the use of a compound selected from: gaboxadol acetate, gaboxadol citrate, gaboxadol fumarate, gaboxadol phosphate, gaboxadol tartrate, gaboxadol succinate, gaboxadol sulfate and gaboxadol bis-sulfate, gaboxadol calcium, gaboxadol potassium, gaboxadol magnesium, gaboxadol sodium, gaboxadol choline, gaboxadol L-lysine and gaboxadol N,N-dibenzyl(ethylene)diamine, or a hydrate, solvate or polymorphic form thereof, for the manufacture of a medicament for treatment of disorders susceptible to amelioration by GABAA receptor agonism which comprising combining such compound with a pharmaceutical carrier or diluent.

In a particular embodiment of the invention, the disorder is susceptible to amelioration by agonism of GABA receptors comprising α4 and δ subunits.

In a further embodiment of the invention, the disorder is selected from neurological or psychiatric disorders such as epilepsy, Parkinson's disease, schizophrenia and Huntington's disease; sleep disorders such as insomnia; premenstrual syndrome; hearing disorders such as tinnitus; vestibular disorders such as Meniere's disease; attention deficit/hyperactivity disorder; intention tremor; and restless leg syndrome.

In a still further embodiment of the invention, the disorder is a sleep disorder, in particular insomnia such as primary insomnia, chronic insomnia or transient insomnia. Within this embodiment is provided the use of the compounds of this invention for increasing total sleep time, increasing non-REM (rapid eye movement) sleep time and/or decreasing sleep latency.

The compounds of this invention may be administered to patients in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. A typical dose is in the range from about 5 mg to about 50 mg per adult person per day, e.g. 5 mg, 10 mg, 15 mg, 20 mg or 25 mg daily. The pharmaceutical composition is preferably provided in a solid dosage formulation comprising about 1 mg, 5 mg, 10 mg, 15 mg, 20 mg, 25 mg or 50 mg active ingredient.

The X-ray powder diffraction spectra was generated on a Philips Analytical X'Pert PRO X-ray Diffraction System with PW3040/60 console. A PW3373/00 ceramic Cu LEF X-ray tube K-Alpha radiation was used as the source. The X-ray powder diffraction spectrum was recorded at ambient temperature (CuKα radiation, 3° to 40° (2θ), steps of 0.014°, 0.2 sec per step), giving the results herein. Solid-state carbon-13 NMR spectrum was obtained on a Bruker DSX 400WB NMR system using a Bruker 4 mm double resonance CPMAS probe. Carbon-13 NMR spectrum utilized proton/carbon-13 cross-polarization magic-angle spinning with variable-amplitude cross polarization. The sample was spun at 15.0 kHz, and a total of 2048 scans were collected with a recycle delay of 20 seconds. A line broadening of 40 Hz was applied to the spectrum before FT was performed. Chemical shifts are reported on the TMS scale using the carbonyl carbon of glycine (176.03 p.p.m.) as a secondary reference. DSC traces were recorded between 25 and 300° C. (10° C./min), under a flow of dry nitrogen. TGA was carried out between 25 and 300° C. (10° C./min), under a flow of dry nitrogen.

### Example 1

### Preparation of Gaboxadol Acid Salts

A 25 mg/ml solution of gaboxadol was prepared by dissolving 1.25 g gaboxadol in 50 ml of water and manually dispensed 400 uL of this substrate stock solution to each of the 96 wells in the plate, resulting in 10 mg (0.071 mmol) of substrate per well. Following the substrate dispense, 9 different 0.1M acid stock solutions were dispensed in columns on the 96 well plate, one mole equivalent of each acid and column 6 was charged with 0.5 mole equivalent of sulfuric acid. After the substrate and acids were dispensed to each of the wells, the 96-well plate was placed in the centrifugal evaporator to remove all the solvents. The plate was evaporated for 2.5 hours at 1300 rpm, 35° C., under 1-8 mbar pressure. Acetic acid was charged to column 1 and hydrochloric acid was then added to two columns on the plate as a 1.0M solution in diethyl ether (1 mole equivalent to column 8, 2 mole equivalents to column 9). This was followed by a solvent dispense, manually charged in rows (800 uL/well).

The mapping of the 96-well plate is as follows:

Acid mapping (down columns)
Column 1: Acetic Acid
Column 2: L-Ascorbic Acid
Column 3: Sulfuric Acid (1 equiv.)
Column 4: Citric Acid
Column 5: Fumaric Acid
Column 6: Sulfuric Acid (0.5 equiv.)
Column 7: Phosphoric Acid
Column 8: Hydrochloric Acid (1 equiv.)
Column 9: Hydrochloric Acid (2 equiv.)
Column 10: L-Tartaric Acid
Column 11: Succinic Acid
Column 12: Maleic Acid
Crystallization solvent mapping (800 uL)
Row A: Ethanol
Row B: 2-Propanol
Row C: 1,2-Dichloroethane
Row D: Trifluorotoluene
Row E: Isopropyl Acetate
Row F: Nitromethane
Row G: Acetonitrile
Row H: 1,2-Dimethoxyethane

Once all the solvents were dispensed to each well, the 96-well plate was capped and heated to 60 deg C. for 2 hours. The plate was then daughtered twice, 400 uL into an evaporation plate and 400 uL into a cooling plate. The cooling plate was cooled with a cubic cool down temperature gradient of 65-10° C. over 10 hours and the evaporation plate was allowed to dry overnight. Each experiment was wicked to remove the remaining solvent and the plates were removed for analysis. The wells were inspected both visually and by polarized light microscopy for birefringence. Wells containing birefringent material were then scanned by XRPD. XRPD patterns were obtained for all of the wells with crystals. The XRPD patterns were then compared to each other, to known forms of gaboxadol and the acids and bases used in the crystallization experiments. They were sorted into groups based on their similarities. Salts formed with HCl were all determined to be the same as the known form and were not explored further. Based on the sorted XRPD patterns, salts with novel patterns were scaled up for further characterization by birefringence, XRPD, DSC and TGA.

### Example 2

Preparation of Gaboxadol Base Salts A 25 mg/ml solution of gaboxadol was prepared by dissolving 1.25 g gaboxadol in 50 ml of water and manually dispensed 400 uL of this substrate stock solution to each of the 96 wells in the plate, resulting in 10 mg (0.071 mmol) of substrate per well. Following the substrate dispense, 9 different 0.1M base stock solutions were dispensed in columns on the 96 well plate (magnesium hydroxide and calcium hydroxide were added manually as powders). After the substrate and bases were dispensed to each of the wells, the 96-well plate was placed in the centrifugal evaporator to remove all the solvents. The plate was evaporated for 2.5 hours at 1300 rpm, 35° C., under 1-8 mbar pressure. Ammonium hydroxide was then added to the plate as a 1.0M solution. This was followed by a solvent dispense, manually charged in rows (800 uL/well).

The mapping of the 96-well plate is as follows:

Base mapping (down columns)
Column 1: Potassium Hydroxide
Column 2: Sodium Hydroxide
Column 3: Choline Hydroxide
Column 4: L-Lysine
Column 5: L-Arginine
Column 6: N-methyl-D-Glucamine
Column 7: Tris (Hydroxymethyl) Aminomethane
Column 8: Magnesium Hydroxide
Column 9: Ammonium Hydroxide
Column 10: Ethanolamine
Column 11: N,N′-Dibenzyl(ethylene)diamine
Column 12: Calcium Hydroxide
Crystallization solvent mapping (800 uL)
Row A: Ethanol
Row B: 2-Propanol
Row C: Water
Row D: Isopropyl Acetate
Row E: Acetonitrile
Row F: Nitromethane
Row G: 1,2-Dimethoxyethane
Row H: 1,2-Dichloroethane

Once all the solvents were dispensed to each well, the 96-well plate was capped and heated to 60 deg C. for 2 hours. The plate was then daughtered twice, 400 uL into an evaporation plate and 400 uL into a cooling plate. The cooling plate was cooled with a cubic cool down temperature gradient of 65-10° C. over 10 hours and the evaporation plate was allowed to dry overnight. Each experiment was wicked to remove the remaining solvent and the plates were removed for analysis. The wells were inspected both visually and by polarized light microscopy for birefringence. Wells containing birefringent material were then scanned by XRPD. XRPD patterns were obtained for all of the wells with crystals. The XRPD patterns were then compared to each other, to known forms of gaboxadol and the acids and bases used in the crystallization experiments. They were sorted into groups based on their similarities. Salts formed with HCl were all determined to be the same as the known form and were not explored further. Based on the sorted XRPD patterns, salts with novel patterns were scaled up for further characterization by birefringence, XRPD, DSC and TGA.

### Example 3

### General Procedure for Preparation of Gaboxadol Acid Salts

Several experiments were scaled to 100 mg. Gaboxadol was charged as a 25 mg/mL solution in water and acid was added neat. The mixtures were dried and then reslurried in recrystallization solvent. For experiments from the cooling plate the vials were thermal cycled with a cubic cool down temperature gradient of 65-10° C. over 10 hours as in the screening plates. Those experiments from the evaporation plate were left open to evaporate for several days. Solids from the cooling experiments were filtered and these solids and those from the evaporation experiments were analyzed by birefringence, x-ray powder diffraction, DSC and TGA.

### Example 4

### Preparation of Gaboxadol Acetate Salt

Using the procedure of Example 3, 1 mole equivalent of acetic acid was added neat (40.8 uL), crystallized in both trifluorotoluene and isopropyl acetate by evaporation (2 forms). Upon scale-up analysis by XRPD shows the crystallization from triflurotoluene produced the form initially observed from crystallization with isopropyl acetate. Screening plate results also indicated the formation of the acetate salt by cooling and in two other solvents (nitromethane and 1,2-dichloroethane).

Acetate Salt: DSC: endotherm onset at 109° C. with exothermic transition onset at 204° C., TGA: 5.0 wt % loss from 40° C. to 115° C. followed by exothermic decomposition onset at 205° C.

### Example 5

### Preparation of Gaboxadol Sulfate Salt (1 Mole Equivalent)

Using the procedure of Example 3, 1 mole equivalent of sulfuric acid was added as a 1.0M solution in water (713 uL) and the water was removed by centrifugal evaporation. The sulfate salt was crystallized in acetonitrile by evaporation. Upon scale-up the XRPD pattern matched the expected predominant pattern produced in the screen. Screening plate results also indicate the formation of the sulfate salt by evaporation and in several other solvents (2-propanol, trifluorotoluene, isopropylacetate, nitromethane, 1,2-dimethoxyethane and ethanol). From the cooling plate, the crystallization from ethanol produced a form also occurring from an experiment with only 0.5 equivalent of sulfuric acid crystallized in both nitromethane and acetonitrile (Type III). Sulfate Salt (1 mol equiv.): DSC: two endotherms 1.) Onset at 119° C. and 2.) Onset at 162° C. with exothermic decomposition onset at about 190° C., TGA: 7.1 wt % loss from 50° C. to 176° C. followed by decomposition onset at 196° C.

### Example 6

### Preparation of Gaboxadol Citrate Salt

Using the procedure of Example 3, 1 mole equivalent of citric acid was added neat (137.09 mg), crystallized in three different solvents: 2-propanol, isopropyl acetate and acetonitrile all by evaporation. Upon scale-up the XRPD produced new patterns not observed in the screen. The scale-ups from 2-propanol and acetonitrile are similar to each other. The scale-up crystallization from isopropyl acetate appears to be a different phase by XRPD. Citrate Salt from 2-propanol and acetonitrile: DSC: endotherm onset at 162° C. with exothermic decomposition onset at 175° C., TGA: 8.9 wt % loss from 32° C. to 103° C. followed by decomposition onset at about 190° C. Citrate Salt from isopropyl acetate: DSC: endotherm onset at 164° C. with exothermic transition onset at 175° C., TGA: 18.5 wt % loss from 31° C. to 109° C. followed by decomposition onset at about 190° C.

### Example 7

Preparation of Gaboxadol Fumarate Salt Using the procedure of Example 3, 1 mole equivalent of fumaric acid was added neat (82.82 mg), recrystallized in ethanol by evaporation. Upon scale-up the XRPD produced a pattern that matched the form produced in the screen. Screening plate results also indicate the formation of the fumarate salt by cooling and in several other solvents (2-propanol, 1,2-dichloroethane, trifluorotoluene, isopropyl acetate, nitromethane, acetonitrile and 1,2-dimethoxyethane). Fumarate Salt: DSC: exothermic decomposition onset at 215° C., TGA: no weight loss, decomposition onset at about 190° C.

### Example 8

### Preparation of Gaboxadol Sulfate Salt (0.5 Mole Equivalent)

Three forms were identified from the screen with 0.5 equivalents of sulfuric acid that were not completely consistent for each solvent when comparing the evaporation and cooling plates.

| | | |
|---|---|---|
| Type I | Type II | Type III |

Evaporation (Scaled up)

| | | |
|---|---|---|
| Row A (ethanol) | Row C (1,2-dichloroethane) | Row F (nitromethane) |
| Row B (2-propanol) | Row D (trifluorotoluene) | Row G (acetonitrile) |
| Row H (1,2-di- | Row E (isopropylacetate) | |
| methoxyethane) | | |

Cooling (Scaled up)

| | |
|---|---|
| Row C (1,2-dichloroethane) | Row A (ethanol) |
| Row D (trifluorotoluene) | Row B (2-propanol) |
| | Row F (nitromethane) |
| | Row G (acetonitrile) |
| | Row H (1,2-di- |
| | methoxyethane) |

Using the procedure of Example 3, 0.5 mole equivalent of sulfuric acid was added as a 1.0M solution in water (357 uL) and the water was removed by centrifugal evaporation. The possible bis-sulfate salt was crystallized in three different solvents: ethanol (Type I) by evaporation and trifluorotoluene (Type II) and acetonitrile (Type III) by cooling. Upon scale-up the XRPD patterns did not all match the expected patterns produced in the screen. The pattern produced from crystallization with both ethanol (expected Type I) and acetonitrile (expected Type III) produced Type III. Crystallization with triflurotoluene produced solids that exhibit an XRPD pattern matching Type I (expected Type II). Sulfate Salt from trifluorotoluene (Type I): DSC: endotherm onset at 166° C. with exothermic decomposition onset at 220° C., TGA: two steps 1.) 1.2 wt % loss from about 50° C. to 87° C. 2.) 4.3 wt % loss from 87° C. to 180° C. followed by decomposition onset at about 220° C. Sulfate Salt from ethanol and acetonitrile (Type III): DSC: two endotherms 1.) Onset at 138° C. and 2.) Onset at 180° C. with exothermic decomposition onset at 207° C., TGA: two steps 1.) 1.4 wt % loss from about 50° C. to 118° C. 2.) 0.8 wt % loss from 118° C. to 154° C. followed by decomposition onset at about 220° C.

### Example 9

### Preparation of Gaboxadol Phosphate Salt

Using the procedure of Example 3, 1 mole equivalent of phosphoric acid was added as a 1.0M solution in water (713 uL) and the water was removed by centrifugal evaporation. The sulfate salt was crystallized in ethanol by cooling. Upon scale-up the XRPD pattern matches the expected pattern produced in the screen. Screening plate results also indicate the formation of the phosphate salt by evaporation and in several other solvents (2-propanol, 1,2-dichloroethane, trifluorotoluene, isopropyl acetate, nitromethane, 1,2-dimethoxyethane and acetonitrile). Phosphate Salt: DSC: exothermic decomposition onset at about 200° C., TGA: no weight loss, decomposition onset at 214° C.

### Example 10

### Preparation of Gaboxadol Tartrate Salt

Using the procedure of Example 3, 1 mole equivalent of L-Tartaric Acid was added neat (107.1 mg), crystallized in ethanol by evaporation (Type I) and 2-propanol (Type II) by cooling. Upon scale-up the solids recovered from both experiments produced the same new XRPD pattern (Type III) and another pattern. Screening plate results also indicate the formation of the tartrate salt Type I by evaporation in 2-propanol, 1,2-dichloroethane, isopropyl acetate and nitromethane and by cooling in 1,2-dichloroethane and nitromethane. Additionally, Type II was identified on the cooling plate from ethanol. L-Tartrate Salt: DSC: endotherm onset at 192° C. with exothermic decomposition immediately following at 199° C., TGA: 0.6 wt % loss from 31° C. to 177° C. followed by decomposition onset at about 189° C.

### Example 11

### Preparation of Gaboxadol Succinate Salt

Using the procedure of Example 3, 1 mole equivalent of Succinic acid was added neat (84 mg), crystallized in ethanol by cooling. Upon scale-up the XRPD pattern matches the expected pattern produced in the screen. Screening plate results also indicate the formation of the succinate salt by evaporation and cooling in several other solvents (2-propanol, 1,2-dichloroethane, trifluorotoluene, isopropyl acetate, nitromethane, 1,2-dimethoxyethane and acetonitrile). Succinate Salt: DSC: exothermic decomposition onset at 198° C., TGA: no weight loss, decomposition onset at 193° C.

### Example 12

### General Procedure for Preparation of Gaboxadol Base Salts

Several experiments were scaled to 100 mg. Gaboxadol was charged as a 25 mg/mL solution in water and base was added neat followed by recrystallization solvent. For experiments from the cooling plate the vials were thermal cycled with a cubic cool down temperature gradient of 65-10° C. over 10 hours. Those experiments from the evaporation plate were left open to evaporate for several days. Solids were filtered and analyzed by birefringence, x-ray powder diffraction, DSC and TG.

### Example 13

### Preparation of Gaboxadol Potassium Salt

Using the procedure of Example 12, 1 molar equivalent of potassium hydroxide was added neat (40.0 mg), crystallized in 1,2-dichloroethane by cooling. Upon scale-up the vial was held at 65° C. overnight instead of cooling. The solids created from this error indicate the formation of another form so the experiment was reproduced. When the crystallization was rerun the XRPD pattern shows the formation of yet another form. Potassium Salt: Both solid forms exhibited exothermic transitions upon analysis by DSC.

### Example 14

### Preparation of Gaboxadol Sodium Salt

Using the procedure of Example 12, 1 molar equivalent of sodium hydroxide was added neat (28.5 mg), crystallized in acetonitrile by cooling. Screening plate results also indicate the formation of the sodium salt by evaporation and in several other solvents (2-propanol, water, isopropylacetate, nitromethane, 1,2-dimethoxyethane and 1,2-dichloroethane). Upon scale-up the vial was held at 65° C. overnight instead of cooling. The solids created from this error indicate the formation of another form so the experiment was reproduced. When rerun the XRPD pattern matches the pattern produced in the screen. Sodium Salt: Both solids exhibited exothermic transitions upon analysis by DSC.

### Example 15

### Preparation of Gaboxadol Choline Salt

Using the procedure of Example 12, 1 molar equivalent of choline hydroxide was added neat (86.5 uL), crystallized in 1,2-dichloroethane by cooling. Upon scale-up the XRPD produced a pattern that matched the form produced in the screen. Choline Salt: The solid exhibited exothermic transitions upon analysis by DSC.

### Example 16

### Preparation of Gaboxadol L-Lysine Salt

Using the procedure of Example 12, 1 molar equivalent of L-Lysine was added as a 0. 1M solution in water (7.14 mL), the water was removed and the salt was recrystallized in 1,2-dimethoxyethane by evaporation. Upon scale-up the XRPD produced a pattern that matched the form produced in the screen. L-Lysine Salt: The solid exhibited exothermic transitions upon analysis by DSC.

### Example 17

### Preparation of Gaboxadol Magnesium Salt

Using the procedure of Example 12, 1 molar equivalent of magnesium hydroxide was added neat (41.6 mg), crystallized in 2-propanol by both evaporation and cooling. Screening plate results also indicate the formation of the magnesium salt by evaporation and cooling in isopropylacetate. Upon scale-up the XRPD produced a pattern that matched a mixture of gaboxadol and bulk magnesium hydroxide, not the salt. Another form was produced from evaporation with water. A scale-up experiment was run with 1 molar equivalent of magnesium hydroxide added neat (41.6 mg), crystallized in water by evaporation. After five days the slurry was filtered. The solids recovered produced the same XRPD pattern seen above (crystallized from both 2-propanol and isopropylacetate). Magnesium Salt: DSC analysis did not indicate any exothermic events below a temperature of approximately 345° C.

### Example 18

### Preparation of Gaboxadol Ammonium Salt

Using the procedure of Example 12, 1 molar equivalent of ammonium hydroxide was added as a 23 wt % solution in water (109 uL), crystallized in 2-propanol by evaporation. Screening plate results also indicate the formation of the ammonium salt by evaporation and cooling in acetonitrile, 1,2-dimethoxyethane and 1,2-dichloroethane.

### Example 19

### Preparation of Gaboxadol N,N-dibenzyl(ethylene)diamine Salt

Using the procedure of Example 12, 1 molar equivalent of N,N-dibenzyl(ethylene)diamine was added neat (168.1 uL), crystallized in 2-propanol by both evaporation and cooling. Screening plate results also indicate the formation of the N,N-dibenzyl(ethylene)diamine salt by evaporation and cooling in isopropylacetate and nitromethane. Upon scale-up large crystalline plates were created and the XRPD produced a pattern that matched the form produced in the screen. Another form was produced from evaporation with water. A scale-up experiment was run with 1 molar equivalent of N,N-dibenzyl(ethylene)diamine added neat (168.1 uL), crystallized in water by evaporation. After five days the slurry was filtered. The solids recovered produced the same XRPD pattern produced in the screen. N,N-Dibenzyl-ethylenediamine Salt: The solid exhibited an exothermic transition upon analysis by DSC.

### Example 20

### Preparation of Gaboxadol Calcium Salt

Using the procedure of Example 12, 1 molar equivalent of calcium hydroxide was added neat (52.9 mg), crystallized in water by evaporation. After five days the slurry was filtered. The solids recovered produce the same XRPD pattern produced in the screen. Calcium Salt: The solid exhibited exothermic decomposition on analysis by DSC.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound which is selected from the group consisting of:
gaboxadol acetate, gaboxadol citrate, gaboxadol fumarate, gaboxadol phosphate, gaboxadol tartrate, gaboxadol succinate, gaboxadol sulfate, and gaboxadol bis-sulfate, or a hydrate, solvate or polymorphic form thereof.

2. The compound of claim 1 in crystalline form.

3. The compound of claim 1 which is gaboxadol sulfate, or a hydrate, solvate or polymorphic form thereof.

4. The compound of claim 1 which is gaboxadol bis-sulfate, or a hydrate, solvate or polymorphic form thereof.

5. A compound which is selected from the group consisting of:
gaboxadol calcium, gaboxadol potassium, gaboxadol magnesium, gaboxadol sodium, gaboxadol choline, gaboxadol L-lysine and gaboxadol N,N-dibenzyl(ethylene)diamine, or a hydrate, solvate or polymorphic form thereof.

6. The compound of claim 5 in crystalline form.

7. The compound of claim 5 which is gaboxadol calcium, or a hydrate, solvate or polymorphic form thereof.

8. The compound of claim 5 which is gaboxadol magnesium, or a hydrate, solvate or polymorphic form thereof.

9. A pharmaceutical composition comprising the compound of claim 1 and a pharmaceutically acceptable carrier.

10. A pharmaceutical composition comprising the compound of claim 5 and a pharmaceutically acceptable carrier.

11. (canceled)

12. (canceled)

13. A method for treating epilepsy; Parkinson's disease; schizophrenia; Huntington's disease; sleep disorder; premenstrual syndrome; hearing disorder; vestibular disorder; attention deficit/hyperactivity disorder; intention tremor; or restless leg syndrome, which comprises administering to the patient a therapeutically effective amount of the compound of claim 1.

14. A method for treating epilepsy; Parkinson's disease; schizophrenia; Huntington's disease; sleep disorder; premenstrual syndrome; hearing disorder; vestibular disorder; attention deficit/hyperactivity disorder; intention tremor; or restless leg syndrome, which comprises administering to the patient a therapeutically effective amount of the compound of claim 5.
